(19) United States
(12) Patent Application Publication
(10) Pub. No.: US 2025/0182857 A1
(43) Pub. Date: Jun. 5, 2025

(54) IMMUNOGENICITY PREDICTION MODEL TRAINING METHOD AND APPARATUS, DEVICE, AND STORAGE MEDIUM

(71) Applicant: Tencent Technology (Shenzhen) Company Limited, Shenzhen (CN)

(72) Inventors: Bing HE, Shenzhen (CN); Yixin GUO, Shenzhen (CN); Jianhua YAO, Shenzhen (CN); Yu ZHAO, Shenzhen (CN)

(73) Assignee: Tencent Technology (Shenzhen) Company Limited, Shenzhen (CN)

(21) Appl. No.: 19/043,848

(22) Filed: Feb. 3, 2025

Related U.S. Application Data
(63) Continuation of application No. PCT/CN2023/130705, filed on Nov. 9, 2023.

(30) Foreign Application Priority Data
Jan. 17, 2023 (CN) 202310118750.8

(51) Int. Cl. G16B 40/20; G06N 3/04; G06N 3/09; G16B 20/30

(52) U.S. Cl. CPC G16B 40/20; G06N 3/04; G06N 3/09; G16B 20/30

(57) ABSTRACT

An immunogenicity prediction model training method includes constructing an immunogenicity prediction model that includes a binding prediction sub-model, a presentation prediction sub-model, and an immunogenicity prediction sub-model, the binding prediction sub-model being configured to predict binding between an epitope and a major histocompatibility complex (MHC), the presentation prediction sub-model being configured to predict cell membrane presentation of an antigenic peptide-major histocompatibility complex (pMHC) molecular complex, and the immunogenicity prediction sub-model being configured to predict immunogenicity of the pMHC; inputting a first sample pair into the immunogenicity prediction model to obtain a sample immunogenicity prediction result, the first sample pair including a first sample epitope sequence and a first sample MHC sequence, and the first sample pair being provided with a first sample immunogenicity label; and training the immunogenicity prediction model with the first sample immunogenicity label as a supervisor of the sample immunogenicity prediction result.

### CROSS-REFERENCE TO RELATED APPLICATIONS

This application is a continuation application of International Application No. PCT/CN2023/130705 filed on Nov. 9, 2023, which claims priority to Chinese Patent Application No. 202310118750.8, filed with the China National Intellectual Property Administration on Jan. 17, 2023, the disclosures of each being incorporated by reference herein in their entireties.

### FIELD

The disclosure relates to the field of machine learning, and in particular, to an immunogenicity prediction model training method and apparatus, a device, and a storage medium.

### BACKGROUND

Immunogenicity prediction of epitopes is crucial for vaccine design and T cell therapy.

In the related art, since T cell receptors (TCRs) cannot directly recognize epitopes on the surface of protein antigens, antigenic peptide-major histocompatibility complex (pMHC) molecular complexes may be predicted by logistic regression, or immunogenicity prediction may be performed by learning sequence features of MHCs and peptides through a convolutional neural network.

However, there are a small quantity of pMHCs with immunogenicity labels, which leads to a poor learning effect of a neural network, resulting in low accuracy of immunogenicity predicted by using the neural network.

### SUMMARY

Provided are an immunogenicity prediction model training method and apparatus, a device, and a storage medium.

According to an aspect of the disclosure, an immunogenicity prediction model training method, performed by a computer device, includes constructing an immunogenicity prediction model, the immunogenicity prediction model including a binding prediction sub-model, a presentation prediction sub-model, and an immunogenicity prediction sub-model, the binding prediction sub-model being configured to predict binding between an epitope and a major histocompatibility complex (MHC), the presentation prediction sub-model being configured to predict cell membrane presentation of an antigenic peptide-major histocompatibility complex (pMHC) molecular complex, and the immunogenicity prediction sub-model being configured to predict immunogenicity of the pMHC; inputting a first sample pair into the immunogenicity prediction model to obtain a sample immunogenicity prediction result output by the immunogenicity prediction model, the first sample pair including a first sample epitope sequence and a first sample MHC sequence, and the first sample pair being provided with a first sample immunogenicity label; and generating a trained immunogenicity prediction model by training the immunogenicity prediction model with the first sample immunogenicity label as a supervisor of the sample immunogenicity prediction result.

According to an aspect of the disclosure, an immunogenicity prediction model training apparatus includes at least one memory configured to store computer program code; and at least one processor configured to read the program code and operate as instructed by the program code, the program code including model construction code configured to cause at least one of the at least one processor to construct an immunogenicity prediction model, the immunogenicity prediction model including a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and first training code configured to cause at least one of the at least one processor to input a first sample pair into the immunogenicity prediction model to obtain a sample immunogenicity prediction result output by the immunogenicity prediction model, the first sample pair including a first sample epitope sequence and a first sample MHC sequence, and the first sample pair being provided with a first sample immunogenicity label; and second training code configured to cause at least one of the at least one processor to generate a trained immunogenicity prediction model by training the immunogenicity prediction model with the first sample immunogenicity label as a supervisor of the sample immunogenicity prediction result.

According to an aspect of the disclosure, a non-transitory computer-readable storage medium, storing computer code which, when executed by at least one processor, causes the at least one processor to at least construct an immunogenicity prediction model, the immunogenicity prediction model including a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and input a first sample pair into the immunogenicity prediction model to obtain a sample immunogenicity prediction result output by the immunogenicity prediction model, the first sample pair including a first sample epitope sequence and a first sample MHC sequence, and the first sample pair being provided with a first sample immunogenicity label; and generate a trained immunogenicity prediction model by training the immunogenicity prediction model with the first sample immunogenicity label as a supervisor of the sample immunogenicity prediction result.

### BRIEF DESCRIPTION OF THE DRAWINGS

To describe the technical solutions of some embodiments of this disclosure more clearly, the following briefly introduces the accompanying drawings for describing some embodiments. The accompanying drawings in the following description show only some embodiments of the disclosure, and a person of ordinary skill in the art may still derive other drawings from these accompanying drawings without creative efforts. In addition, one of ordinary skill would understand that aspects of some embodiments may be combined together or implemented alone.

FIG. 1 is a flowchart of an immunogenicity prediction model training method according to some embodiments.

FIG. 2 is a schematic diagram of a T cell immunogenicity formation process and corresponding sub-models according to some embodiments.

FIG. 3 is a flowchart of a process of training an immunogenicity prediction model according to some embodiments.

FIG. 4 is a schematic diagram of a structure of an epitope sequence feature encoder according to some embodiments.

FIG. 5 is a schematic diagram of an immunogenicity prediction model according to some embodiments.

FIG. 6 is a flowchart of an immunogenicity prediction method according to some embodiments.

FIG. 7 is a block diagram of a structure of an immunogenicity prediction model training apparatus according to some embodiments.

FIG. 8 is a block diagram of a structure of an immunogenicity prediction apparatus according to some embodiments.

FIG. 9 is a schematic diagram of a structure of a computer device according to some embodiments.

### DESCRIPTION OF EMBODIMENTS

To make the objectives, technical solutions, and advantages of the present disclosure clearer, the following further describes the present disclosure in detail with reference to the accompanying drawings. The described embodiments are not to be construed as a limitation to the present disclosure. All other embodiments obtained by a person of ordinary skill in the art without creative efforts shall fall within the protection scope of the present disclosure.

In the following descriptions, related “some embodiments” describe a subset of all possible embodiments. However, it may be understood that the “some embodiments” may be the same subset or different subsets of all the possible embodiments, and may be combined with each other without conflict. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. For example, the phrase “at least one of A, B, and C” includes within its scope “only A”, “only B”, “only C”, “A and B”, “B and C”, “A and C” and “all of A, B, and C.”

For ease of understanding, some terms involved in some embodiments are explained and described below.

Immunogenicity: the characteristic for an antigen or epitope to act on an antigen recognition receptor of T cells or B cells to induce a body to generate a humoral or cell-mediated immune response.

For T cells, an antigen that can induce the T cells to generate an immune response is a peptide presented by an MHC to the surface of the cells, for example, a pMHC.

Peptide: a compound formed from three or more amino acids linked by peptide bonds, also an intermediate product in protein hydrolysis.

Peptide bond: a chemical bond formed through dehydration and condensation of amino and carboxyl groups between amino acid molecules.

Major histocompatibility complex (MHC): a protein for binding a peptide and presenting the peptide as an antigen to the surface of cells. A pMHC complex formed by binding the MHC to the peptide can bind a T cell receptor on the surface of the cells to activate T cells, so as to induce a T-cell-mediated immune response.

Antigen: a substance that can activate a body to generate an immune response and can extracellularly bind immune response products and sensitized lymphocytes to produce an immune effect.

Epitope: also referred to as an antigenic determinant, is a site on an antigen molecule at which an immune cell receptor is bound. T cells can only recognize a pMHC presented by an MHC, and the pMHC is obtained by binding the epitope to the MHC.

T cell receptor: a receptor on the surface of T cells that is responsible for recognizing an antigen presented by an MHC to activate an immune response.

In the process of inducing a T cell immune response, an MHC binds a peptide to obtain a pMHC, and presenting the pMHC to the surface of the cells as an antigen to bind the T cell receptor, to induce the T-cell-mediated immune response.

For T cells, a pMHC that can trigger the T cells to generate an immune response has immunogenicity, and the strength of the immunogenicity determines the ability of the pMHC sequence to activate the immune response of the T cells. Currently, only a small quantity of pMHCs have immunogenicity labels, and a large quantity of pMHCs have unknown immunogenicity. In the process of vaccine design and development, vaccines made based on pMHCs with stronger immunogenicity can better activate immune responses in the body, and whether a pMHC has immunogenicity may be determined in immunization and protection against diseases.

In the related art, there are a small quantity of pMHCs with immunogenicity labels and defects in a model structure, which leads to poor performance of a model for directly predicting immunogenicity, with an area under curve (AUC) remaining at 0.7 or 0.8.

Therefore, some embodiments provide an immunogenicity prediction model training method. The use of the model breaks the limit of the small quantity of pMHCs with immunogenicity labels, and predicts immunogenicity of a pMHC more accurately, with an AUC reaching 0.9.

The immunogenicity prediction model training method and the immunogenicity prediction method according to some embodiments may be performed by a computer device. The computer device may be an electronic device with strong computing performance, such as a personal computer, a workstation, or a server. A type of the computer device is not limited.

In an application scenario, after training of an immunogenicity prediction model is completed by using a training device, the immunogenicity prediction model obtained through training may be deployed in a computer or server, and subsequently immunogenicity prediction is performed in the computer or server using the immunogenicity prediction model.

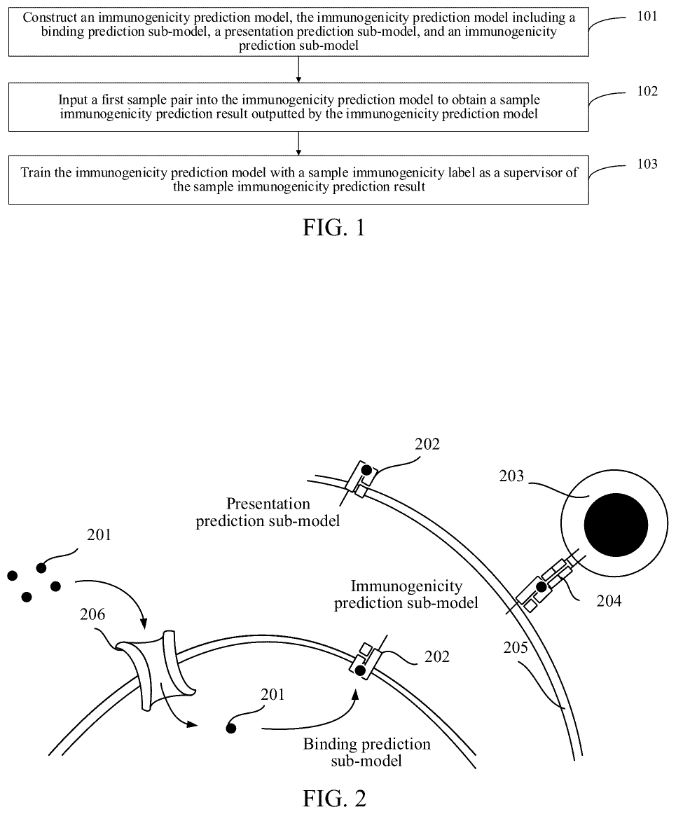

FIG. 1 is a flowchart of an immunogenicity prediction model training method according to some embodiments. An example in which the method is performed by a computer device is used for description in some embodiments. The method includes the following operations.

Operation 101: Construct an immunogenicity prediction model, the immunogenicity prediction model including a binding prediction sub-model, a presentation prediction sub-model, and an immunogenicity prediction sub-model.

The binding prediction sub-model is configured to predict binding between an epitope and an MHC, the presentation prediction sub-model is configured to predict cell membrane presentation of a pMHC, and the immunogenicity prediction sub-model is configured to predict immunogenicity of the pMHC. For example, the foregoing three sub-models respectively correspond to three stages of a T cell immunogenicity formation process, and the constructed immunogenicity prediction model is configured to predict immunogenicity of a pMHC with a combination of three biologically relevant tasks.

Binding is the property of binding between the epitope and the MHC to form a pMHC. During an immune response, a MHC can selectively bind an epitope with specificity, and not any epitope can bind an MHC. The binding prediction sub-model is configured to predict a probability of success in binding between the epitope and the MHC. The probability of success may be a probability in a 0/1 distribution, where 0 indicates that the binding is not possible, and 1 indicates that the binding is possible. The probability of success may be any value in the interval [0, 1].

Presentation is the property of the pMHC, obtained by binding the epitope to the MHC, to be presented to the surface of cell membranes. The MHC is the main antigen-presenting molecule in the immune system. T cells cannot recognize a pure antigen, but can recognize an antigen bound to the antigen-presenting molecule on the surface of cells. The pMHC is obtained by binding the epitope to the MHC molecule. The pMHC obtained through binding is presented through cell membranes to the surface of the cells for recognition by the TCR. The presentation prediction sub-model is configured to predict a probability of success in presentation of the pMHC to the surface of cell membranes. The probability of success may be a probability in a 0/1 distribution, where 0 indicates that the presentation to the surface of cell membranes is not possible, and 1 indicates that the presentation to the surface of cell membranes is possible. The probability of success may be any value in the interval [0, 1].

Immunogenicity is the property to induce an immune response by T cells. Only some pMHCs have immunogenicity. The immune response can be initiated after the pMHC is recognized by the TCR on the surface of T cells. The immunogenicity prediction sub-model is configured to predict a probability of success in induction of an immune response by binding the pMHC to the T cell receptor, for example, a probability that the pMHC has immunogenicity. The probability that the pMHC has immunogenicity may be a probability in a 0/1 distribution, where 0 indicates that the pMHC does not have immunogenicity, and 1 indicates that the pMHC has immunogenicity. The probability that the pMHC has immunogenicity may be any value in the interval [0, 1].

However, since there are a small quantity of pMHCs with immunogenicity labels, a single immunogenicity prediction sub-model has a poor learning effect, resulting in low accuracy of an immunogenicity prediction result of a pMHC. In some embodiments, the biologically relevant binding prediction sub-model and presentation prediction sub-model are introduced to construct the immunogenicity prediction model together with the immunogenicity prediction sub-model, which breaks the limit of the small quantity of pMHCs with immunogenicity labels by multi-task learning, making the performance of the immunogenicity prediction model better.

Operation 102: Input a first sample pair into the immunogenicity prediction model to obtain a sample immunogenicity prediction result output by the immunogenicity prediction model.

The first sample pair includes a first sample epitope sequence (for representing a first sample epitope) and a first sample MHC sequence (for representing a first sample MHC), and the first sample pair is provided with a sample immunogenicity label. The sample immunogenicity label is configured to indicate whether a pMHC formed by the first sample epitope sequence and the first sample MHC sequence has immunogenicity.

In some embodiments, a sample immunogenicity label 1 indicates that the pMHC formed by the first sample epitope and the first sample MHC has immunogenicity, and a sample immunogenicity label 0 indicates that the pMHC sequence formed by the first sample epitope and the first sample MHC does not have immunogenicity.

In some embodiments, the immunogenicity prediction model is trained by using the first sample pair with the sample immunogenicity label, and the first sample epitope sequence included in each first sample pair can bind the first sample MHC sequence to form the pMHC sequence.

After the first sample pair is inputted into the to-be-trained immunogenicity prediction model, the output sample immunogenicity prediction result has a certain difference from a result indicated by the sample immunogenicity label. The input of the first sample pair with the sample immunogenicity label into the immunogenicity prediction model enables the immunogenicity prediction model to continuously learn and implement classification of input data based on the sample immunogenicity label of the first sample pair.

Operation 103: Train the immunogenicity prediction model with the sample immunogenicity label as a supervisor of the sample immunogenicity prediction result.

Initially, the sample immunogenicity prediction result output by the immunogenicity prediction model has a large difference from the sample immunogenicity label. During model training, the computer device fine-tunes a parameter in the immunogenicity prediction model, to make the difference between the sample immunogenicity prediction result and the sample immunogenicity label gradually reduced.

The computer device fine-tunes the immunogenicity prediction model in the direction of reducing the difference between the sample immunogenicity prediction result and the sample immunogenicity label, with the sample immunogenicity label as the supervisor of the sample immunogenicity prediction result.

In some embodiments, the computer device determines a prediction loss based on the sample immunogenicity prediction result and the sample immunogenicity label, to train the immunogenicity prediction model based on the prediction loss.

In some embodiments, the computer device determines a probability difference based on an immunogenicity probability represented by the sample immunogenicity prediction result and an immunogenicity probability true value represented by the sample immunogenicity label, to train the immunogenicity prediction model with the probability difference as the prediction loss.

In some embodiments, in a case that the prediction loss converges or a quantity of rounds of training is reached, the computer device completes the model training.

In some embodiments, the construction of the immunogenicity prediction model based on the binding prediction sub-model, the presentation prediction sub-model, and the immunogenicity prediction sub-model is based on a T cell immunogenicity formation process, introducing, based on a single immunogenicity prediction task, a binding prediction task and a presentation prediction task that are biologically relevant. The introduction of multiple biologically relevant tasks enables the model to learn feature expression of the epitope sequence and the MHC sequence more efficiently, improving quality of model training with a limited pMHC dataset (pMHCs with immunogenicity labels). Further, immunogenicity prediction is performed on an unknown pMHC by using the immunogenicity prediction model obtained through training, which can improve accuracy of an immunogenicity prediction result.

FIG. 2 is a schematic diagram of a T cell immunogenicity formation process and corresponding sub-models according to some embodiments. In this figure, a transporter 206 associated with antigen processing transports an antigenic peptide 201, for example, an epitope, into a lumen of an endoplasmic reticulum, and the antigenic peptide 201 binds an MHC in the lumen of the endoplasmic reticulum to form a pMHC 202. A binding prediction sub-model is configured to predict a probability of binding between the antigenic peptide 201 and the MHC to form the pMHC 202. Then, the pMHC 202 is transported through a cell membrane 205 to the surface of a cell. A presentation prediction sub-model is configured to predict a probability of transportation of the pMHC 202 to the surface of the cell. Finally, a T cell receptor 204 on the surface of a cell membrane of a T cell 203 binds the pMHC 202 to induce an immune response. An immunogenicity prediction sub-model is configured to predict a probability of induction of the immune response by binding the T cell receptor 204 to the pMHC 202.

In the process of T cell immunogenicity formation of the pMHC, each operation corresponds to one sub-model, so that an immunogenicity prediction model can predict immunogenicity of the pMHC with the help of three biologically relevant tasks.

In some embodiments, the immunogenicity prediction model includes the binding prediction sub-model, the presentation prediction sub-model, and the immunogenicity prediction sub-model, the three sub-models share inputs, and outputs of the binding prediction sub-model, the presentation prediction sub-model, the immunogenicity prediction sub-model are connected to the same fully connected layer, and finally the fully connected layer outputs an immunogenicity prediction result.

The three sub-models share inputs. For example, a first sample pair inputted into the immunogenicity prediction model is separately inputted into the binding prediction sub-model, the presentation prediction sub-model, and the immunogenicity prediction sub-model to obtain three different data features, respectively corresponding to a binding feature, a presentation feature, and an immunogenicity feature of input data. Outputs of the sub-models are connected to the same fully connected layer, which can calculate a probability that the inputted pMHC has immunogenicity.

During training, after inputting the first sample pair into the immunogenicity prediction model, the computer device inputs the first sample pair separately into the binding prediction sub-model, the presentation prediction sub-model, and the immunogenicity prediction sub-model, to obtain a first sample pair feature, a second sample pair feature, and a third sample pair feature. The first sample pair feature is the binding feature output by the binding prediction sub-model. The second sample pair feature is the presentation feature output by the presentation prediction sub-model. The third sample pair feature is the immunogenicity feature output by the immunogenicity prediction sub-model.

After obtaining the first sample pair feature, the second sample pair feature, and the third sample pair feature, the computer device performs feature fusion on the first sample pair feature, the second sample pair feature, and the third sample pair feature to obtain a fused sample pair feature.

In some embodiments, the feature fusion may be performed by weighted fusion (for example, assigning different weights to different features and performing weighted summation on the features), feature concatenation (for example, concatenating multiple features sequentially to obtain a longer feature), or feature stacking (for example, stacking multiple features in a manner to obtain a new feature, where the stacking may be an addition or subtraction operation, or may be a complex operation such as convolution or pooling). A manner of feature fusion is not limited.

In some embodiments, the computer device first performs feature fusion (such as weighted fusion or feature stacking) on the first sample pair feature and the second sample pair feature to obtain an intermediate fused feature of binding and presentation, and then performs feature fusion (for example, feature concatenation) on the intermediate fused feature and the third sample pair feature to obtain the fused sample pair feature.

Further, the computer device inputs the fused sample pair feature into the fully connected layer to obtain a sample immunogenicity prediction result output by the fully connected layer.

The fully connected layer can play a role in mapping distributed feature representation learned by the model to sample labeling space, for example, mapping the fused sample pair feature output by the three sub-models to the sample labeling space, to finally obtain the sample immunogenicity prediction result. In some embodiments, the fully connected layer may be implemented by a convolution operation.

In some embodiments, for the immunogenicity prediction model, classification may be performed by softmax logistic regression.

In some embodiments, the computer device may pass, through two fully connected layers with sigmoid activation functions, the first sample pair feature, the second sample pair feature, and the third sample pair feature that are output by the three sub-models, to generate a final prediction result. The first fully connected layer is configured to perform linear combination on the features. The second fully connected layer is configured to perform highly non-linear transformation on data inputted into the fully connected layer.

In some embodiments, the computer device passes, through one fully connected multi-layer perceptron, the first sample pair feature, the second sample pair feature, and the third sample pair feature that are output by the three sub-models, to obtain the sample immunogenicity prediction result output by the immunogenicity prediction model.

In some embodiments, when training the immunogenicity prediction model, the computer device fine-tunes parameters of the binding prediction sub-model, the presentation prediction sub-model, the immunogenicity prediction sub-model, and the fully connected layer respectively based on a prediction loss of the model.

The immunogenicity prediction model is constructed based on the trained binding prediction sub-model, presentation prediction sub-model, and immunogenicity prediction sub-model. Before the immunogenicity prediction model is constructed, the binding prediction sub-model, the presentation prediction sub-model, and the immunogenicity prediction sub-model have to be trained separately.

In some embodiments, the computer device trains the binding prediction sub-model based on a second sample pair. The second sample pair includes a second sample epitope sequence and a second sample MHC sequence, and the second sample pair is provided with a sample binding label. The sample binding label is configured to indicate whether a second sample epitope can bind a second sample MHC to form a pMHC. In some embodiments, the sample binding label is a 0/1 label, where 0 indicates that the second sample epitope cannot bind the second sample MHC to form the pMHC, and 1 indicates that the second sample epitope can bind the second sample MHC to form the pMHC.

In some embodiments, the computer device inputs the second sample pair into the binding prediction sub-model to obtain a second sample binding prediction result, and trains the binding prediction sub-model with the sample binding label as a supervisor of the second sample binding prediction result.

In some embodiments, the computer device trains the presentation prediction sub-model based on a third sample pair. The third sample pair includes a third sample epitope sequence and a third sample MHC sequence, and the third sample pair is provided with a sample presentation label. The sample presentation label is configured to indicate whether a pMHC formed by a third sample epitope and a third sample MHC can be presented to the surface of cells. In some embodiments, the sample presentation label may be a 0/1 label, where 0 indicates that the pMHC formed by the third sample epitope and the third sample MHC cannot be presented to the surface of cells, and 1 indicates that the pMHC formed by the third sample epitope and the third sample MHC can be presented to the surface of cells.

In some embodiments, the computer device inputs the third sample pair into the presentation prediction sub-model to obtain a third sample presentation prediction result, and trains the presentation prediction sub-model with the sample presentation label as a supervisor of the third sample presentation prediction result.

In some embodiments, the computer device trains the immunogenicity prediction sub-model based on a fourth sample pair. The fourth sample pair includes a fourth sample epitope sequence and a fourth sample MHC sequence, and the fourth sample pair is provided with a sample immunogenicity label. The sample immunogenicity label is configured to indicate whether a pMHC formed by a fourth sample epitope and a fourth sample MHC has immunogenicity. In some embodiments, the sample immunogenicity label may be a 0/1 label, where 0 indicates that the pMHC formed by the fourth sample epitope and the fourth sample MHC does not have immunogenicity, and 1 indicates that the pMHC formed by the fourth sample epitope and the fourth sample MHC has immunogenicity.

In some embodiments, the computer device inputs the fourth sample pair into the immunogenicity prediction sub-model to obtain a fourth sample immunogenicity prediction result, and trains the immunogenicity prediction sub-model with the sample immunogenicity label as a supervisor of the fourth sample immunogenicity prediction result.

The binding prediction sub-model and the presentation prediction sub-model are trained respectively using the second sample pair and the third sample pair, which can enable the binding prediction sub-model and the presentation prediction sub-model to better represent binding and presentation of an epitope sequence and an MHC sequence. As there are a large quantity of sample pairs with binding labels and presentation labels, the binding prediction sub-model and the presentation prediction sub-model have better training effects. The immunogenicity prediction model is then constructed based on the trained binding prediction sub-model and presentation prediction sub-model with the immunogenicity prediction sub-model, which can improve the performance of the immunogenicity prediction model.

In the process of training the immunogenicity prediction model, after constructing the immunogenicity prediction model based on the binding prediction sub-model, the presentation prediction sub-model, and the immunogenicity prediction sub-model that are obtained through training, the computer device may input the first sample pair into the immunogenicity prediction model to train the immunogenicity prediction model.

In some embodiments, since the first sample pair has the sample immunogenicity label and the fourth sample pair for training the immunogenicity prediction sub-model is also provided with the sample immunogenicity label, the fourth sample pair may be inputted as the first sample pair into the immunogenicity prediction model to train the immunogenicity prediction model.

To further improve the performance of the model, the computer device may augment training data by self-distillation, and train the model based on augmented training data.

In some embodiments, before inputting the first sample pair into the immunogenicity prediction model, the computer device obtains a prediction result of a fifth sample pair based on the immunogenicity prediction model, and determines an immunogenicity pseudo label corresponding to the fifth sample pair and a prediction confidence level.

The fifth sample pair has no sample immunogenicity label, for example, whether the fifth sample pair has immunogenicity is unknown. After the fifth sample pair is inputted into the immunogenicity prediction model, the immunogenicity prediction model outputs a probability that the fifth sample pair has immunogenicity. In addition, the computer device determines the immunogenicity pseudo label corresponding to the fifth sample pair based on the probability that the fifth sample pair has immunogenicity and the prediction confidence level.

After determining the immunogenicity pseudo label, the computer device selects, from the fifth sample pair, a sample pair with the prediction confidence level higher than a confidence level threshold. The confidence level threshold may be set according to a case. For example, the confidence level threshold may be set to 0.9. When a confidence level is higher than the immunogenicity threshold, a prediction result may be considered highly accurate. If the prediction result of the fifth sample pair indicates that the fifth sample pair has immunogenicity, and the prediction confidence level is high, the fifth sample pair is more likely to have immunogenicity.

Before the training of the immunogenicity prediction model is completed, a part of the prediction result obtained based on the fifth sample pair has a low confidence level. The computer device selects a sample pair with a confidence level higher than the confidence level threshold, and generates the first sample pair from the selected fifth sample pair and the fourth sample pair. Because the computer device determines the pseudo label of the fifth sample pair through calculation of the immunogenicity prediction model, and the selected fifth sample pair has a high confidence level, the pseudo label of the selected fifth sample pair may be used as the sample immunogenicity label of the first sample pair. In other words, the first sample pair includes the fourth sample pair with the sample immunogenicity label and the part of the fifth sample pair with the immunogenicity pseudo label.

In some embodiments, after constructing the immunogenicity prediction model based on the binding prediction sub-model, the presentation prediction sub-model, and the immunogenicity prediction sub-model that are obtained through training, the computer device trains the immunogenicity prediction model based on the first sample pair including only the fourth sample pair. After one round of training, the computer device inputs the fifth sample pair into the immunogenicity prediction model, and the computer device can determine the immunogenicity pseudo label corresponding to the fifth sample pair and the prediction confidence level based on the prediction result output by the immunogenicity prediction model. Then, the fifth sample pair with a high confidence level is selected and added into the first sample pair. The first sample pair includes the fourth sample pair and the fifth sample pair. Finally, the immunogenicity prediction model is trained again based on the reconstituted first sample pair. This manner may be understood as a process of model self-distillation.

In some embodiments, the fifth sample pair with a high confidence level in the prediction result output by the immunogenicity prediction model is added into the first sample pair, and the immunogenicity pseudo label of the fifth sample pair is used as the sample immunogenicity label of the first sample pair to supervise the training of the immunogenicity prediction model, which can increase a quantity of training data for training the immunogenicity prediction model, so that the immunogenicity prediction model can better learn the feature of the first sample pair, enhancing the representation of the immunogenicity prediction model, and improving the performance of the immunogenicity prediction model.

In the first sample pair, a quantity of sample pairs with immunogenicity is often lower than a quantity of sample pairs without immunogenicity. A sample pair with immunogenicity may be referred to as a positive sample. A sample pair without immunogenicity may be referred to as a negative sample.

When the immunogenicity prediction model is trained by using the first sample pair, due to the imbalance between a positive sample quantity and a negative sample quantity in the first sample pair, the immunogenicity prediction model learns more features of negative samples, resulting in inaccuracy of a final prediction result.

To overcome the problem of model training quality affected by an insufficient positive sample quantity, when training the immunogenicity prediction model with the sample immunogenicity label as a supervisor of the sample immunogenicity prediction result, the computer device trains the immunogenicity prediction model based on a weighted loss. During training, there is a certain difference between the sample immunogenicity prediction result and the sample immunogenicity label since the training of the immunogenicity prediction model is not completed, and the weighted loss may represent the difference between the sample immunogenicity prediction result and the sample immunogenicity label. Training the immunogenicity prediction model with the sample immunogenicity label as the supervisor of the sample immunogenicity prediction result is a process of continuously adjusting a parameter of the immunogenicity prediction model to continuously reduce the weighted loss, for example, to continuously reduce the difference between the sample immunogenicity prediction result and the sample immunogenicity label. When the weighted loss converges, the training of the immunogenicity prediction model is completed.

Training the immunogenicity prediction model based on the weighted loss includes the following operations.

FIG. 3 is a flowchart of a process of training an immunogenicity prediction model according to some embodiments. The process includes the following operations:

Operation 301: Determine a positive sample quantity and a negative sample quantity based on the sample immunogenicity label in the first sample pair.

The first sample pair includes the sample immunogenicity label. The computer device determines the first sample pair with immunogenicity indicated by the sample immunogenicity label as a positive sample, and determines the first sample pair without immunogenicity indicated by the sample immunogenicity label as a negative sample.

After classifying all the first sample pairs based on the sample immunogenicity label, the computer device may determine the positive sample quantity and the negative sample quantity.

Operation 302: Determine a positive sample weight and a negative sample weight based on the positive sample quantity and the negative sample quantity.

A sample weight and a sample quantity are in a positive correlation.

The positive sample weight is a proportion of the positive sample quantity in a total sample quantity, and the negative sample weight is a proportion of the negative sample quantity in the total sample quantity. The positive sample weight and the positive sample quantity are in a positive correlation, and the negative sample weight and the negative sample quantity are in a positive correlation. For example, the sample weight may be determined through the following formula:

$${Weight}_{class} = \frac{X [ class ]}{{\sum }_{i} X [ i ]}$$

In this formula, class represents a class of a sample, for example, a positive sample or a negative sample, and Weight_{class} represents a sample weight of a sample of the class. If the class is a positive sample, Weight_{class} represents a sample weight of the positive sample, X[ ] represents a sample quantity, X[class] represents a quantity of samples of the class, X[i] represents a quantity of samples of an i^{th} class, where i has two values, one is a positive sample, and the other is a negative sample.

Operation 303: Determine a prediction loss between the sample immunogenicity label and the sample immunogenicity prediction result.

In the process of training the immunogenicity prediction model, the prediction loss may be calculated by using a binary cross entropy loss function, a 0-1 loss function, an absolute value loss function, or the like.

The prediction loss is a difference between the sample immunogenicity prediction result and the sample immunogenicity label.

For example, the prediction loss calculated by using a cross entropy loss function is:

$$Loss ( X , class ) = log ( \frac{exp ( X [ class ] )}{{\sum }_{i} exp ( X [ i ] )} )$$

In this formula, Loss represents a prediction loss, class represents a class of a sample, for example, a positive sample or a negative sample, and X [i] represents a quantity of samples of an i^{th} class, where i has two values, one is a positive sample, and the other is a negative sample.

Operation 304: Perform loss weighting on the prediction loss based on the positive sample weight and the negative sample weight to obtain a weighted loss.

When a negative sample quantity is large, a negative sample weight is high, so the weighted loss is more in favor of the negative sample, and the negative sample dominates a direction of gradient updating in the training process, masking the role of the positive sample in training the immunogenicity prediction model. After determining the positive sample weight and the negative sample weight, the computer device performs loss weighting based on the positive sample weight and the negative sample weight.

For example, the weighted loss obtained by performing loss weighting on the loss calculated by using the cross entropy loss function is:

$$Loss ( X , class ) = {Weight}_{class} * log ( \frac{exp ( X [ class ] )}{{\sum }_{i} exp ( X [ i ] )} )$$

The meanings of variables in this formula are the same as those in the foregoing two formulas.

Operation 305: Train the immunogenicity prediction model based on the weighted loss.

The weighted loss may represent, to a certain extent, the difference between the immunogenicity prediction result predicted by the immunogenicity prediction model and the sample immunogenicity label. The process of the computer device training the immunogenicity prediction model based on the weighted loss is a process of adjusting the immunogenicity prediction model in a direction of gradually reducing the weighted loss.

The computer device continuously adjusts the immunogenicity prediction model based on the weighted loss until the weighted loss converges, to complete model training.

The binding prediction sub-model, the presentation prediction sub-model, and the immunogenicity prediction sub-model may also be trained through the foregoing operations. For implementation details, refer to operation 301 to operation 305.

In some embodiments, the computer device determines the positive sample weight and the negative sample weight based on the positive sample quantity and the negative sample quantity in the first sample pair, weights the prediction loss based on the positive sample weight and the negative sample weight to obtain the weighted loss, and trains the immunogenicity prediction model based on the weighted loss, which can resolve the problem of imbalance between positive samples and negative samples in the first sample pair for training the immunogenicity prediction model, so that the trained immunogenicity prediction model has better performance in immunogenicity prediction.

In some embodiments, before training the binding prediction sub-model, the presentation prediction sub-model, and the immunogenicity prediction sub-model, the computer device pre-trains an epitope sequence feature encoder and an MHC sequence feature encoder, so that the epitope sequence feature encoder learns feature expression of an epitope sequence, and the MHC sequence feature encoder learns feature expression of an MHC sequence.

After the pre-training of the feature encoders is completed, the binding prediction sub-model, the presentation prediction sub-model, and the immunogenicity prediction sub-model are constructed separately based on the epitope sequence feature encoder and the MHC sequence feature encoder.

Each sub-model includes an epitope sequence feature encoder, an MHC sequence feature encoder, and a fully connected layer.

In some embodiments, the binding prediction sub-model, the presentation prediction sub-model, and the immunogenicity prediction sub-model each include two fully connected layers. The first fully connected layer is configured to perform linear combination on the features. The second fully connected layer is configured to perform highly non-linear transformation on data inputted into the fully connected layer.

The epitope sequence feature encoder is configured to perform feature extraction on an epitope sequence, and the MHC sequence feature encoder is configured to perform feature extraction on an MHC sequence.

In some embodiments, the epitope sequence feature encoder and the MHC sequence feature encoder are of the same structure.

In some embodiments, the epitope sequence feature encoder and the MHC sequence feature encoder may be encoder parts of models such as a convolutional neural network, a recurrent neural network, a Transformer model, and a BERT model.

In some embodiments, an example in which the epitope sequence feature encoder and the MHC sequence feature encoder are Bert model encoders of the same structure is used to describe the process of pre-training the epitope sequence feature encoder and the MHC sequence feature encoder, but the disclosure is not limited thereto.

In some embodiments, the epitope sequence feature encoder in some embodiments includes 12 layers of encoders, and each layer of encoder includes a self-attention layer and a feedforward neural network layer.

The epitope sequence feature encoder and the MHC sequence feature encoder are pre-trained by using a sample epitope sequence and a sample MHC sequence respectively and by using data included in a public immunization database.

For example, the sample epitope sequence may be obtained from the Immune-Epitope Database and Analysis Resource (IEDB), in a length range of 8 bp to 14 bp (where bp represents a base pair). The sample MHC sequence is an MHC pseudo sequence obtained based on DeepLigand, with a length of 34 bp. The sample epitope sequence and the sample MHC sequence have different lengths, and a maximum length of the epitope sequence is 20. The sample MHC sequence is formatted into a token with a length of 20 amino acids, and then two artificial tokens are added, [CLS] as a prefix and [SEP] as a suffix.

In addition, for the sample epitope sequence and the sample MHC sequence, an amino acid may be considered as a word, and thus the sample sequence is divided into a form of multiple amino acids. For example, CASSIGLNTEAFF may be divided into an input sequence including 12 amino acids.

For pre-training the epitope sequence feature encoder, the computer device masks a sample epitope sequence to obtain a sample masked epitope sequence.

In some embodiments, the computer device randomly masks an inputted amino acid sequence, for example, the sample epitope sequence, in a proportion, for example, the proportion may be 15%, and then trains the epitope sequence feature encoder based on another unmasked amino acid sequence in the sequence to predict the masked amino acid. For example, the computer device partially masks a sample epitope sequence CASSIGLNTEAFF to obtain a masked sequence CASS [MAA] GLNTEAFF, where [MAA] represents the amino acid that is randomly masked, and the trained epitope sequence feature encoder predicts that [MAA] is supposed to be I. One [MAA] may indicate that one amino acid is masked, or may indicate that multiple amino acids are masked.

Subsequently, the computer device inputs the sample masked epitope sequence into a first pre-training model to obtain a first mask prediction result output by the first pre-training model.

The first mask prediction result is a prediction result of a mask position in the sample masked epitope sequence. The first pre-training model includes the epitope sequence feature encoder and a first mask prediction header, and the first pre-training model may be a convolutional neural network, a recurrent neural network, a Transformer model, a Bert model, or the like.

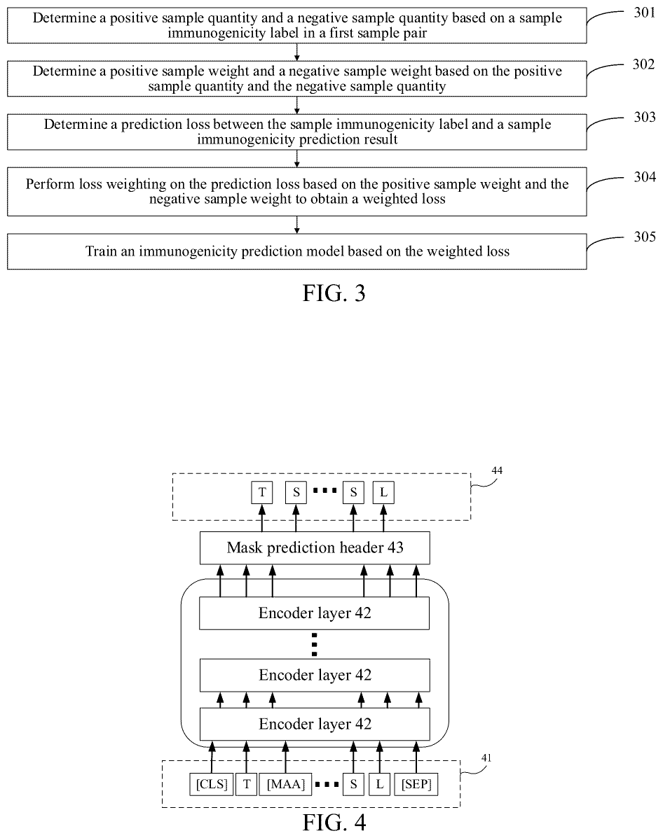

For example, FIG. 4 is a schematic diagram of a structure of a first pre-training model according to some embodiments. A sample epitope sequence 41 that is masked is inputted into the first pre-training model, where the first pre-training model is a Bert model, and encoded through multiple encoder layers 42. Subsequently, different feature parts of amino acids in an amino acid sequence are optimized through a mask prediction header 43, so that richer feature information of the sample epitope sequence can be extracted, improving an effect of feature extraction performed by the epitope sequence feature encoder. Finally, a first mask prediction result 44 is output.

When encoded by the encoder, the sample masked epitope sequence passes through an embedding layer in the first pre-training model, and is then combined with position code of each amino acid, to obtain an initialized representation X of the sample masked epitope sequence. Subsequently, after the initialized representation X is inputted into a self-attention layer, a query vector (Query), a key vector (Key), and a value vector (Value) are first calculated, Q=W^{Q}*X, K=W^{K}*X, V=W^{V}*X, where W^{Q}, W^{K}, and W^{V} are parameter matrices of the initialized representation X mapped to Q, K, and V. Then, a weighted feature vector is calculated:

$$Z = Attention ( Q , K , V ) = softmax ( \frac{{QK}^{T}}{\sqrt{{d}_{k}}} ) V$$

In this formula, dₖ represents a dimension of the query vector Q or the key vector K.

After obtaining the first mask prediction result, the computer device trains the first pre-training model based on the first mask prediction result and actual mask content at a mask position in the sample masked epitope sequence.

In some embodiments, the computer device trains the first pre-training model with the actual mask content at the mask position in the sample masked epitope sequence as a supervisor of the first mask prediction result.

In the process of training the first pre-training model, the computer device calculates a complementary loss based on a difference between the first mask prediction result and the actual mask content at the mask position in the sample masked epitope sequence. The complementary loss may be calculated by using a mean square error loss function, an LI loss, or the like, and a parameter of the first pre-training model is continuously adjusted to make the complementary loss gradually reduced.

For pre-training the MHC sequence feature encoder, the computer device first masks a sample MHC sequence to obtain a sample masked MHC sequence, then inputs the sample masked MHC sequence into a second pre-training model to obtain a second mask prediction result output by the second pre-training model, and finally trains the second pre-training model based on the second mask prediction result and actual mask content at a mask position in the sample masked MHC sequence. The second pre-training model includes the MHC sequence feature encoder and a second mask prediction header.

A process of pre-training the MHC sequence feature encoder is similar to that of pre-training the epitope sequence feature encoder, which may refer to the process of training the first pre-training model.

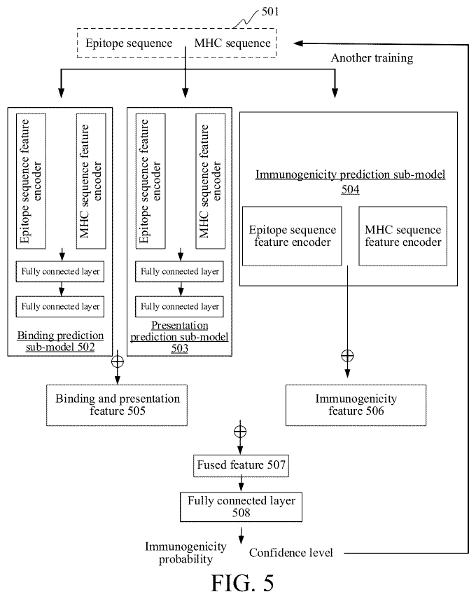

FIG. 5 is a schematic diagram of an immunogenicity prediction model according to some embodiments. During training, an input is a first sample pair 501, for example, a sample epitope sequence and a sample MHC sequence, and the first sample pair 501 is inputted separately into a binding prediction sub-model 502, a presentation prediction sub-model 503, and an immunogenicity prediction sub-model 504. Each sub-model includes one MHC sequence feature encoder, one epitope sequence feature encoder, and two fully connected layers. After the binding prediction sub-model 502, the presentation prediction sub-model 503, and the immunogenicity prediction sub-model 504 respectively extract a first sample pair feature (a binding feature), a second sample pair feature (a presentation feature), and a third sample pair feature (an immunogenicity feature), the first sample pair feature and the second sample pair feature are first combined to obtain a binding and presentation feature 505, and feature fusion is then performed on the binding and presentation feature 505 and the immunogenicity feature 506 to obtain a fused feature 507. Finally, the fused feature is inputted into a fully connected layer 508 to finally obtain an immunogenicity prediction result output by the fully connected layer, including an immunogenicity probability and a confidence level. During self-distillation, the computer device adds a sample pair with a confidence level higher than a confidence level threshold into the first sample pair, to train the immunogenicity prediction model.

The immunogenicity prediction model obtained through training in some embodiments may be configured to predict immunogenicity of a pMHC sequence. The immunogenicity prediction method according to some embodiments is completed based on the immunogenicity prediction model obtained through training in some embodiments.

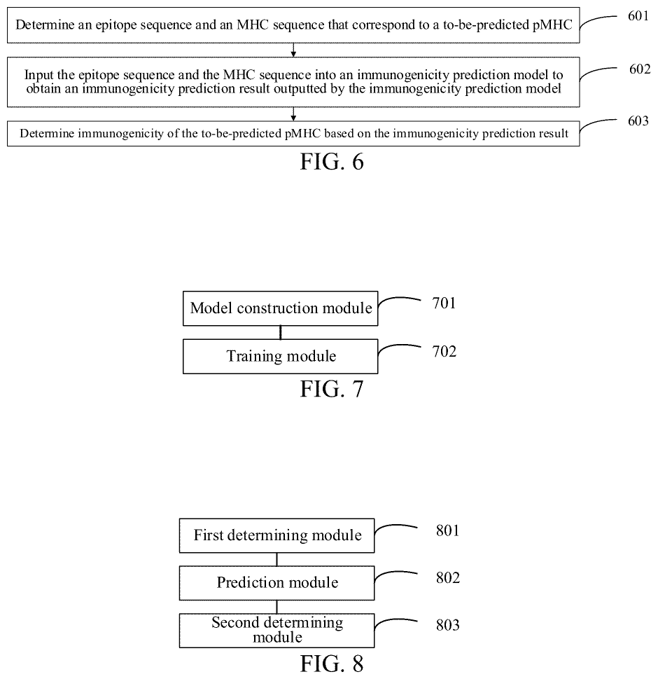

FIG. 6 is a flowchart of an immunogenicity prediction method according to some embodiments. An example in which the method is performed by a computer device is used for description in some embodiments. The method includes the following operations.

Operation 601: Determine an epitope sequence and an MHC sequence that correspond to a to-be-predicted pMHC.

An immunogenicity prediction model used in some embodiments includes a binding prediction sub-model, a presentation prediction sub-model, and an immunogenicity prediction sub-model, and each sub-model includes an epitope sequence feature encoder and an MHC sequence feature encoder. The epitope sequence feature encoder is configured to perform feature encoding on an epitope sequence, and the MHC sequence feature encoder is configured to perform feature encoding on an MHC sequence.

The computer device determines the epitope sequence and the MHC sequence based on the to-be-predicted pMHC, and predicts immunogenicity based on the epitope sequence and the MHC sequence that correspond to the to-be-predicted pMHC.

In some embodiments, the computer device splits the to-be-predicted pMHC to obtain the epitope sequence and the MHC sequence.

Operation 602: Input the epitope sequence and the MHC sequence into an immunogenicity prediction model to obtain an immunogenicity prediction result output by the immunogenicity prediction model.

The immunogenicity prediction model includes a binding prediction sub-model, a presentation prediction sub-model, and an immunogenicity prediction sub-model, the binding prediction sub-model is configured to predict binding between an epitope sequence and an MHC sequence, the presentation prediction sub-model is configured to predict cell membrane presentation of a pMHC, and the immunogenicity prediction sub-model is configured to predict immunogenicity of the pMHC.

After inputting the epitope sequence and the MHC sequence into the immunogenicity prediction model, the computer device inputs the epitope sequence and the MHC sequence separately into the binding prediction sub-model, the presentation prediction sub-model, and the immunogenicity prediction sub-model, connects outputs of the three sub-models through a fully connected layer, and outputs the immunogenicity prediction result.

Operation 603: Determine immunogenicity of the to-be-predicted pMHC based on the immunogenicity prediction result.

The immunogenicity prediction result is a probability that the to-be-predicted pMHC has immunogenicity. The computer device determines the immunogenicity of the to-be-predicted pMHC based on the probability.

In some embodiments, a probability threshold is set in the computer device. In a case that the immunogenicity prediction result is greater than the probability threshold, it is determined that the predicted pMHC has immunogenicity. In a case that the immunogenicity prediction result is less than the probability threshold, it is determined that the predicted pMHC does not have immunogenicity.

In some embodiments, the computer device receives a probability threshold set by a user according to an application scenario. In an application scenario with a high requirement for immunogenicity, the probability threshold is increased correspondingly. In a case that the immunogenicity prediction result is greater than the probability threshold, it is determined that the to-be-predicted pMHC has immunogenicity. In a case that the immunogenicity prediction result is less than the probability threshold, it is determined that the to-be-predicted pMHC does not have immunogenicity.

In some embodiments, the computer device predicts immunogenicity of the to-be-predicted pMHC based on the immunogenicity prediction model obtained through training. Because the immunogenicity prediction model includes the binding prediction sub-model, the presentation prediction sub-model, and the immunogenicity prediction sub-model, which breaks the limit of a small quantity of pMHCs with immunogenicity labels by multi-task learning during training, the immunogenicity prediction model obtained through training has high performance. The immunogenicity prediction model is used in some embodiments to predict immunogenicity of the to-be-predicted pMHC, so the output prediction result has high accuracy.

The binding prediction sub-model, the presentation prediction sub-model, and the immunogenicity prediction sub-model in the immunogenicity prediction model used in some embodiments share inputs, and outputs of the binding prediction sub-model, the presentation prediction sub-model, and the immunogenicity prediction sub-model are connected to a same fully connected layer. The three sub-models share inputs. For example, the to-be-predicted pMHC inputted into the immunogenicity prediction model is separately inputted into the binding prediction sub-model, the presentation prediction sub-model, and the immunogenicity prediction sub-model to obtain three different data features, respectively corresponding to a binding feature, a presentation feature, and an immunogenicity feature of the to-be-predicted pMHC. Outputs of the sub-models are connected to the same fully connected layer, which can calculate a probability that the inputted pMHC has immunogenicity.

For an architecture of the immunogenicity prediction model, refer to FIG. 5. Details are not described again in some embodiments.

After the epitope sequence and the MHC sequence are inputted into the immunogenicity prediction model, the epitope sequence and the MHC sequence are inputted separately into the binding prediction sub-model, the presentation prediction sub-model, and the immunogenicity prediction sub-model to obtain a first sequence feature, a second sequence feature, and a third sequence feature.

Subsequently, feature fusion is performed on the first sequence feature, the second sequence feature, and the third sequence feature to obtain a fused sequence pair feature. Finally, the fused sequence pair feature is inputted into the fully connected layer to obtain the immunogenicity prediction result output by the fully connected layer.

The fully connected layer can play a role in mapping distributed feature representation to sample labeling space, for example, mapping the fused sample pair feature output by the three sub-models to the sample labeling space, to finally obtain the sample immunogenicity prediction result. In some embodiments, the fully connected layer may be implemented by a convolution operation.

In addition, for the immunogenicity prediction model provided in some embodiments, classification may be performed by softmax logistic regression.

In some embodiments, the first sequence feature, the second sequence feature, and the third sequence feature that are output by the three sub-models may be passed through two fully connected layers with sigmoid activation functions, to generate a final prediction result. The first fully connected layer is configured to perform linear combination on the features. The second fully connected layer is configured to perform highly non-linear transformation on data inputted into the fully connected layer.

FIG. 7 is a block diagram of a structure of an immunogenicity prediction model training apparatus according to some embodiments. The apparatus includes:
a model construction module 701, configured to construct an immunogenicity prediction model, the immunogenicity prediction model including a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and
a training module 702, configured to input a first sample pair into the immunogenicity prediction model to obtain a sample immunogenicity prediction result output by the immunogenicity prediction model, the first sample pair including a first sample epitope sequence and a first sample MHC sequence, and the first sample pair being provided with a sample immunogenicity label;
the training module 702 being further configured to train the immunogenicity prediction model with the sample immunogenicity label as a supervisor of the sample immunogenicity prediction result.

In some embodiments, the binding prediction sub-model, the presentation prediction sub-model, and the immunogenicity prediction sub-model in the immunogenicity prediction model share inputs, and outputs of the binding prediction sub-model, the presentation prediction sub-model, and the immunogenicity prediction sub-model are connected to a same fully connected layer.

In some embodiments, the training module 702 is configured to:
input the first sample pair separately into the binding prediction sub-model, the presentation prediction sub-model, and the immunogenicity prediction sub-model to obtain a first sample pair feature, a second sample pair feature, and a third sample pair feature, the first sample pair feature being output by the binding prediction sub-model, the second sample pair feature being output by the presentation prediction sub-model, and the third sample pair feature being output by the immunogenicity prediction sub-model;
perform feature fusion on the first sample pair feature, the second sample pair feature, and the third sample pair feature to obtain a fused sample pair feature; and
input the fused sample pair feature into the fully connected layer to obtain the sample immunogenicity prediction result output by the fully connected layer.

In some embodiments, the training module 702 is further configured to:
train the binding prediction sub-model based on a second sample pair, the second sample pair including a second sample epitope sequence and a second sample MHC sequence, and the second sample pair being provided with a sample binding label;
train the presentation prediction sub-model based on a third sample pair, the third sample pair including a third sample epitope sequence and a third sample MHC sequence, and the third sample pair being provided with a sample presentation label; and
train the immunogenicity prediction sub-model based on a fourth sample pair, the fourth sample pair including a fourth sample epitope sequence and a fourth sample MHC sequence, and the fourth sample pair being provided with a sample immunogenicity label.

In some embodiments, the training module 702 is further configured to:
determine, based on a prediction result of the immunogenicity prediction model for a fifth sample pair, an immunogenicity pseudo label corresponding to the fifth sample pair and a prediction confidence level, the fifth sample pair having no sample immunogenicity label;
select, from the fifth sample pair, a sample pair with the prediction confidence level higher than a confidence level threshold; and
generate the first sample pair based on the selected fifth sample pair and the fourth sample pair, a sample immunogenicity label of the selected fifth sample pair being the immunogenicity pseudo label.

In some embodiments, the training module 702 is further configured to:
determine a positive sample quantity and a negative sample quantity based on the sample immunogenicity label in the first sample pair;
determine a positive sample weight and a negative sample weight based on the positive sample quantity and the negative sample quantity, a sample weight and a sample quantity being in a positive correlation;
determine a prediction loss between the sample immunogenicity label and the sample immunogenicity prediction result;
perform loss weighting on the prediction loss based on the positive sample weight and the negative sample weight to obtain a weighted loss; and
train the immunogenicity prediction model based on the weighted loss.

In some embodiments, the apparatus further includes:
a pre-training module, configured to pre-train an epitope sequence feature encoder and an MHC sequence feature encoder, the epitope sequence feature encoder being configured to perform feature extraction on an epitope sequence, and the MHC sequence feature encoder being configured to perform feature extraction on an MHC sequence.

The pre-training module is further configured to construct the binding prediction sub-model, the presentation prediction sub-model, and the immunogenicity prediction sub-model separately based on the epitope sequence feature encoder and the MHC sequence feature encoder, the binding prediction sub-model, the presentation prediction sub-model, and the immunogenicity prediction sub-model each including the epitope sequence feature encoder, the MHC sequence feature encoder, and a fully connected layer.

In some embodiments, the pre-training module is configured to:
mask a sample epitope sequence to obtain a sample masked epitope sequence; input the sample masked epitope sequence into a first pre-training model to obtain a first mask prediction result output by the first pre-training model, the first mask prediction result being a prediction result of a mask position in the sample masked epitope sequence, and the first pre-training model including the epitope sequence feature encoder and a first mask prediction header; and train the first pre-training model based on the first mask prediction result and actual mask content at the mask position in the sample masked epitope sequence; and
mask a sample MHC sequence to obtain a sample masked MHC sequence; input the sample masked MHC sequence into a second pre-training model to obtain a second mask prediction result output by the second pre-training model, the second mask prediction result being a prediction result of a mask position in the sample masked MHC sequence, and the second pre-training model including the MHC sequence feature encoder and a second mask prediction header; and train the second pre-training model based on the second mask prediction result and actual mask content at the mask position in the sample masked MHC sequence.

In some embodiments, the construction of the immunogenicity prediction model based on the binding prediction sub-model, the presentation prediction sub-model, and the immunogenicity prediction sub-model is based on a T cell immunogenicity formation process, introducing, based on a single immunogenicity prediction task, a binding prediction task and a presentation prediction task that are biologically relevant. The introduction of multiple biologically relevant tasks enables the model to learn feature expression of the epitope sequence and the MHC sequence more efficiently, improving quality of model training with a limited pMHC dataset (pMHCs with immunogenicity labels). Further, immunogenicity prediction is performed on an unknown pMHC by using the immunogenicity prediction model obtained through training, which can improve accuracy of an immunogenicity prediction result.

FIG. 8 is a block diagram of a structure of an immunogenicity prediction apparatus according to some embodiments. As shown in FIG. 8, the apparatus includes:
a first determining module 801, configured to determine an epitope sequence and an MHC sequence that correspond to a to-be-predicted pMHC;
a prediction module 802, configured to input the epitope sequence and the MHC sequence into an immunogenicity prediction model to obtain an immunogenicity prediction result output by the immunogenicity prediction model, the immunogenicity prediction model including a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and
a second determining module 803, configured to determine immunogenicity of the to-be-predicted pMHC based on the immunogenicity prediction result.

In some embodiments, the binding prediction sub-model, the presentation prediction sub-model, and the immunogenicity prediction sub-model in the immunogenicity prediction model share inputs, and outputs of the binding prediction sub-model, the presentation prediction sub-model, and the immunogenicity prediction sub-model are connected to a same fully connected layer.

In some embodiments, the prediction module 802 is configured to:
input the epitope sequence and the MHC sequence separately into the binding prediction sub-model, the presentation prediction sub-model, and the immunogenicity prediction sub-model to obtain a first sequence feature, a second sequence feature, and a third sequence feature;
perform feature fusion on the first sequence feature, the second sequence feature, and the third sequence feature to obtain a fused sequence pair feature; and
input the fused sequence pair feature into the fully connected layer to obtain the immunogenicity prediction result output by the fully connected layer.

In some embodiments, the computer device predicts immunogenicity of the to-be-predicted pMHC based on the immunogenicity prediction model obtained through training. Because the immunogenicity prediction model includes the binding prediction sub-model, the presentation prediction sub-model, and the immunogenicity prediction sub-model, which breaks the limit of a small quantity of pMHCs with immunogenicity labels by multi-task learning during training, the immunogenicity prediction model obtained through training has high performance. The immunogenicity prediction model is used in some embodiments to predict immunogenicity of the to-be-predicted pMHC, so the output prediction result has high accuracy.

According to some embodiments, each module may exist respectively or be combined into one or more modules. Some modules may be further split into multiple smaller function subunits, thereby implementing the same operations without affecting the technical effects of some embodiments. The modules are divided based on logical functions. In actual applications, a function of one module may be realized by multiple modules, or functions of multiple modules may be realized by one module. In some embodiments, the apparatus may further include other modules. In actual applications, these functions may also be realized cooperatively by the other modules, and may be realized cooperatively by multiple modules.

A person skilled in the art would understand that these “modules” could be implemented by hardware logic, a processor or processors executing computer software code, or a combination of both. The “modules” may also be implemented in software stored in a memory of a computer or a non-transitory computer-readable medium, where the instructions of each module are executable by a processor to thereby cause the processor to perform the respective operations of the corresponding module.

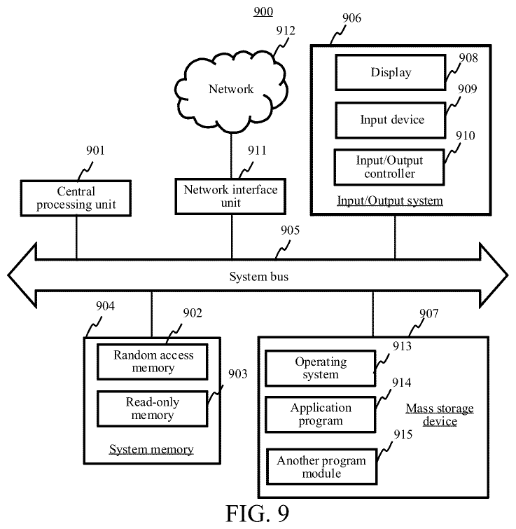

FIG. 9 is a schematic diagram of a structure of a computer device according to some embodiments. The computer device 900 includes a central processing unit (CPU) 901, a system memory 904 including a random access memory 902 and a read-only memory 903, and a system bus 905 connecting the system memory 904 and the central processing unit 901. The computer device 900 further includes an input/output (I/O) system 906 assisting in information transmission between components in the computer, and a mass storage device 907 configured to store an operating system 913, an application program 914, and another program module 915.

In some embodiments, the input/output system 906 includes a display 908 configured to display information, and an input device 909 configured to input information by a user, such as a mouse or a keyboard. The display 908 and the input device 909 are both connected to the central processing unit 901 by using an input/output controller 910 connected to the system bus 905. The input/output system 906 may further include the input/output controller 910 to be configured to receive and process inputs from multiple other devices such as a keyboard, a mouse, and an electronic stylus. The input/output controller 910 further provides an output to a display screen, a printer, or another type of output device.

The mass storage device 907 is connected to the central processing unit 901 by using a mass storage controller connected to the system bus 905. The mass storage device 907 and a computer-readable medium associated with the mass storage device provide non-volatile storage for the computer device 900. In other words, the mass storage device 907 may include a computer-readable medium such as a hard disk or a drive.

Without loss of generality, the computer-readable medium may include a computer storage medium and a communication medium. The computer storage medium includes volatile and non-volatile media, and removable and non-removable media implemented by any method or technique for storing information such as computer-readable instructions, data structures, program modules, or other data. The computer storage medium includes a random access memory (RAM), a read-only memory (ROM), a flash memory or another solid-state storage technique, a compact disc read-only memory (CD-ROM), a digital versatile disc (DVD) or another optical storage, a magnetic cassette, a magnetic tape, a magnetic disk storage, or another magnetic storage device. Certainly, a person skilled in the art may learn that the computer storage medium is not limited to the foregoing several types. The system memory 904 and the mass storage device 907 may be collectively referred to as a memory.

The memory stores one or more programs. The one or more programs are configured to be executed by one or more central processing units 901. The one or more programs include instructions for implementing the foregoing methods. The central processing unit 901 executes the one or more programs to implement the methods provided in some embodiments.

According to some embodiments, the computer device 900 may be further connected, through a network such as the Internet, to a remote computer on the network to run. In other words, the computer device 900 may be connected to a network 912 through a network interface unit 911 connected to the system bus 905, or may be connected to another type of network or a remote computer system through the network interface unit 911.

The memory further includes one or more programs. The one or more programs are stored in the memory. The one or more programs include operations that are configured for performing the methods provided in some embodiments and that are performed by the computer device.

Some embodiments provide a computer program product or a computer program. The computer program product or the computer program includes computer instructions. The computer instructions are stored in a computer-readable storage medium. A processor of a computer device reads the computer instructions from the computer-readable storage medium. The processor executes the computer instructions to enable the computer device to perform the immunogenicity prediction model training method or the immunogenicity prediction method according to the foregoing aspects.

A person of ordinary skill in the art may understand that all or some of the operations in the methods in some embodiments may be implemented by a program instructing relevant hardware. The program may be stored in a computer-readable storage medium. The computer-readable storage medium may be the computer-readable storage medium included in the memory in the foregoing embodiment, or may be a computer-readable storage medium that exists independently and that is not assembled in a terminal. The computer-readable storage medium stores at least one instruction, at least one section of a program, a code set, or an instruction set. The at least one instruction, the at least one section of the program, the code set, or the instruction set is loaded or executed by a processor to implement the immunogenicity prediction model training method or the immunogenicity prediction method according to any one of some embodiments.

In some embodiments, the computer-readable storage medium may include: a ROM, a RAM, a solid state drive (SSD), an optical disc, or the like. The RAM may include a resistance random access memory (ReRAM) and a dynamic random access memory (DRAM). The sequence numbers of some embodiments are merely for description, but do not indicate the preference among embodiments.

A person of ordinary skill in the art may understand that all or some of the operations in some embodiments may be implemented by hardware, or may be implemented by a program instructing relevant hardware. The program may be stored in a computer-readable storage medium. The storage medium may be a read-only memory, a magnetic disk, an optical disc, or the like.

The foregoing embodiments are used for describing, instead of limiting the technical solutions of the disclosure. A person of ordinary skill in the art shall understand that although the disclosure has been described in detail with reference to the foregoing embodiments, modifications can be made to the technical solutions described in the foregoing embodiments, or equivalent replacements can be made to some technical features in the technical solutions, provided that such modifications or replacements do not cause the essence of corresponding technical solutions to depart from the spirit and scope of the technical solutions of the embodiments of the disclosure and the appended claims.

What is claimed is:

1. An immunogenicity prediction model training method, performed by a computer device, the method comprising:
constructing an immunogenicity prediction model, the immunogenicity prediction model comprising a binding prediction sub-model, a presentation prediction sub-model, and an immunogenicity prediction sub-model, the binding prediction sub-model being configured to predict binding between an epitope and a major histocompatibility complex (MHC), the presentation prediction sub-model being configured to predict cell membrane presentation of an antigenic peptide-major histocompatibility complex (pMHC) molecular complex, and the immunogenicity prediction sub-model being configured to predict immunogenicity of the pMHC;
inputting a first sample pair into the immunogenicity prediction model to obtain a sample immunogenicity prediction result output by the immunogenicity prediction model, the first sample pair comprising a first sample epitope sequence and a first sample MHC sequence, and the first sample pair being provided with a first sample immunogenicity label; and
generating a trained immunogenicity prediction model by training the immunogenicity prediction model with the first sample immunogenicity label as a supervisor of the sample immunogenicity prediction result.

2. The immunogenicity prediction model training method according to claim 1, wherein the binding prediction sub-model, the presentation prediction sub-model, and the immunogenicity prediction sub-model share inputs, and outputs of the binding prediction sub-model, the presentation prediction sub-model, and the immunogenicity prediction sub-model are connected to a same fully connected layer.

3. The immunogenicity prediction model training method according to claim 2, wherein the inputting the first sample pair into the immunogenicity prediction model comprises:
inputting the first sample pair separately into the binding prediction sub-model, the presentation prediction sub-model, and the immunogenicity prediction sub-model to obtain a first sample pair feature, a second sample pair feature, and a third sample pair feature, the first sample pair feature being output by the binding prediction sub-model, the second sample pair feature being output by the presentation prediction sub-model, and the third sample pair feature being output by the immunogenicity prediction sub-model;
performing feature fusion on the first sample pair feature, the second sample pair feature, and the third sample pair feature to obtain a fused sample pair feature; and
inputting the fused sample pair feature into the fully connected layer to obtain the sample immunogenicity prediction result output by the fully connected layer.

4. The immunogenicity prediction model training method according to claim 1, wherein before constructing the immunogenicity prediction model, the method further comprises:
training the binding prediction sub-model based on a second sample pair, the second sample pair comprising a second sample epitope sequence and a second sample MHC sequence, and the second sample pair being provided with a sample binding label;
training the presentation prediction sub-model based on a third sample pair, the third sample pair comprising a third sample epitope sequence and a third sample MHC sequence, and the third sample pair being provided with a sample presentation label; and
training the immunogenicity prediction sub-model based on a fourth sample pair, the fourth sample pair comprising a fourth sample epitope sequence and a fourth sample MHC sequence, and the fourth sample pair being provided with a second sample immunogenicity label.

5. The immunogenicity prediction model training method according to claim 4, wherein before inputting the first sample pair into the immunogenicity prediction model, the method further comprises:
determining, based on a first prediction result of the immunogenicity prediction model for a fifth sample pair, an immunogenicity pseudo label corresponding to the fifth sample pair and a prediction confidence level, the fifth sample pair having no sample immunogenicity label;
selecting, from the fifth sample pair, one sample pair with the prediction confidence level higher than a confidence level threshold; and
generating the first sample pair based on the one sample pair and the fourth sample pair, a third sample immunogenicity label of the one sample pair being the immunogenicity pseudo label.

6. The immunogenicity prediction model training method according to claim 5, wherein after generating the first sample pair based on the one sample pair and the fourth sample pair, the method further comprises:
determining a positive sample quantity and a negative sample quantity based on the first sample immunogenicity label; and
determining a positive sample weight and a negative sample weight based on the positive sample quantity and the negative sample quantity, a sample weight and a sample quantity being in a positive correlation, and
wherein the generating the trained immunogenicity prediction model comprises:
determining a prediction loss between the first sample immunogenicity label and the sample immunogenicity prediction result;
performing loss weighting on the prediction loss based on the positive sample weight and the negative sample weight to obtain a weighted loss; and
training the immunogenicity prediction model based on the weighted loss.

7. The immunogenicity prediction model training method according to claim 1, wherein before constructing the immunogenicity prediction model, the method further comprises:
pre-training an epitope sequence feature encoder and an MHC sequence feature encoder, the epitope sequence feature encoder being configured to perform feature extraction on an epitope sequence, and the MHC sequence feature encoder being configured to perform feature extraction on an MHC sequence; and
constructing the binding prediction sub-model, the presentation prediction sub-model, and the immunogenicity prediction sub-model separately based on the epitope sequence feature encoder and the MHC sequence feature encoder, the binding prediction sub-model, the presentation prediction sub-model, and the immunogenicity prediction sub-model each comprising the epitope sequence feature encoder, the MHC sequence feature encoder, and a fully connected layer.

8. The immunogenicity prediction model training method according to claim 7, wherein the pre-training comprises:
masking a sample epitope sequence to obtain a sample masked epitope sequence; inputting the sample masked epitope sequence into a first pre-training model to obtain a first mask prediction result outputted by the first pre-training model, the first mask prediction result being a first prediction result of a first mask position in the sample masked epitope sequence, and the first pre-training model comprising the epitope sequence feature encoder and a first mask prediction header; and training the first pre-training model based on the first mask prediction result and actual mask content at the first mask position; and
masking a sample MHC sequence to obtain a sample masked MHC sequence; inputting the sample masked MHC sequence into a second pre-training model to obtain a second mask prediction result outputted by the second pre-training model, the second mask prediction result being a second prediction result of a second mask position in the sample masked MHC sequence, and the second pre-training model comprising the MHC sequence feature encoder and a second mask prediction header; and training the second pre-training model based on the second mask prediction result and actual mask content at the second mask position.

9. The immunogenicity prediction model training method according to claim 7, wherein the epitope sequence feature encoder and the MHC sequence feature encoder are each based on at least one of: a convolutional neural network, a recurrent neural network, a Transformer model, or a BERT model.

10. The immunogenicity prediction model training method according to claim 7, wherein a plurality of layers of the epitope sequence feature encoder comprise self-attention layers and feedforward neural network layers.

11. An immunogenicity prediction model training apparatus, the apparatus comprising:
at least one memory configured to store computer program code; and
at least one processor configured to read the program code and operate as instructed by the program code, the program code comprising:
model construction code configured to cause at least one of the at least one processor to construct an immunogenicity prediction model, the immunogenicity prediction model comprising a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and
first training code configured to cause at least one of the at least one processor to input a first sample pair into the immunogenicity prediction model to obtain a sample immunogenicity prediction result output by the immunogenicity prediction model, the first sample pair comprising a first sample epitope sequence and a first sample MHC sequence, and the first sample pair being provided with a first sample immunogenicity label; and
second training code configured to cause at least one of the at least one processor to generate a trained immunogenicity prediction model by training the immunogenicity prediction model with the first sample immunogenicity label as a supervisor of the sample immunogenicity prediction result.

12. The immunogenicity prediction model training apparatus according to claim 11, wherein the binding prediction sub-model, the presentation prediction sub-model, and the immunogenicity prediction sub-model share inputs, and outputs of the binding prediction sub-model, the presentation prediction sub-model, and the immunogenicity prediction sub-model are connected to a same fully connected layer.

13. The immunogenicity prediction model training apparatus according to claim 12, wherein the first training code is configured to cause at least one of the at least one processor to:
input the first sample pair separately into the binding prediction sub-model, the presentation prediction sub-model, and the immunogenicity prediction sub-model to obtain a first sample pair feature, a second sample pair feature, and a third sample pair feature, the first sample pair feature being output by the binding prediction sub-model, the second sample pair feature being output by the presentation prediction sub-model, and the third sample pair feature being output by the immunogenicity prediction sub-model;
perform feature fusion on the first sample pair feature, the second sample pair feature, and the third sample pair feature to obtain a fused sample pair feature; and
input the fused sample pair feature into the fully connected layer to obtain the sample immunogenicity prediction result output by the fully connected layer.

14. The immunogenicity prediction model training apparatus according to claim 11, wherein the program code further comprises third training code configured to cause at least one of the at least one processor to:
train the binding prediction sub-model based on a second sample pair, the second sample pair comprising a second sample epitope sequence and a second sample MHC sequence, and the second sample pair being provided with a sample binding label;
train the presentation prediction sub-model based on a third sample pair, the third sample pair comprising a third sample epitope sequence and a third sample MHC sequence, and the third sample pair being provided with a sample presentation label; and
train the immunogenicity prediction sub-model based on a fourth sample pair, the fourth sample pair comprising a fourth sample epitope sequence and a fourth sample MHC sequence, and the fourth sample pair being provided with a second sample immunogenicity label.

15. The immunogenicity prediction model training apparatus according to claim 14, wherein the program code further comprises sample pair generating code configured to cause at least one of the at least one processor to:
determine, based on a first prediction result of the immunogenicity prediction model for a fifth sample pair, an immunogenicity pseudo label corresponding to the fifth sample pair and a prediction confidence level, the fifth sample pair having no sample immunogenicity label;
select, from the fifth sample pair, one sample pair with the prediction confidence level higher than a confidence level threshold; and
generate the first sample pair based on the one sample pair and the fourth sample pair, a third sample immunogenicity label of the one sample pair being the immunogenicity pseudo label.

16. The immunogenicity prediction model training apparatus according to claim 15, wherein the program code further comprises fourth training code configured to cause at least one of the at least one processor to:
determine a positive sample quantity and a negative sample quantity based on the first sample immunogenicity label; and
determine a positive sample weight and a negative sample weight based on the positive sample quantity and the negative sample quantity, a sample weight and a sample quantity being in a positive correlation, and
wherein the second training code is configured to cause at least one of the at least one processor to:
determine a prediction loss between the first sample immunogenicity label and the sample immunogenicity prediction result;
perform loss weighting on the prediction loss based on the positive sample weight and the negative sample weight to obtain a weighted loss; and
train the immunogenicity prediction model based on the weighted loss.

17. The immunogenicity prediction model training apparatus according to claim 11, wherein the program code further comprises constructing code configured to cause at least one of the at least one processor to:
pre-train an epitope sequence feature encoder and an MHC sequence feature encoder, the epitope sequence feature encoder being configured to perform feature extraction on an epitope sequence, and the MHC sequence feature encoder being configured to perform feature extraction on an MHC sequence; and
construct the binding prediction sub-model, the presentation prediction sub-model, and the immunogenicity prediction sub-model separately based on the epitope sequence feature encoder and the MHC sequence feature encoder, the binding prediction sub-model, the presentation prediction sub-model, and the immunogenicity prediction sub-model each comprising the epitope sequence feature encoder, the MHC sequence feature encoder, and a fully connected layer.

18. The immunogenicity prediction model training apparatus according to claim 17, wherein the constructing code is configured to cause at least one of the at least one processor to:
mask a sample epitope sequence to obtain a sample masked epitope sequence; inputting the sample masked epitope sequence into a first pre-training model to obtain a first mask prediction result outputted by the first pre-training model, the first mask prediction result being a first prediction result of a first mask position in the sample masked epitope sequence, and the first pre-training model comprising the epitope sequence feature encoder and a first mask prediction header; and training the first pre-training model based on the first mask prediction result and actual mask content at the first mask position; and
mask a sample MHC sequence to obtain a sample masked MHC sequence; inputting the sample masked MHC sequence into a second pre-training model to obtain a second mask prediction result outputted by the second pre-training model, the second mask prediction result being a second prediction result of a second mask position in the sample masked MHC sequence, and the second pre-training model comprising the MHC sequence feature encoder and a second mask prediction header; and training the second pre-training model based on the second mask prediction result and actual mask content at the second mask position.

19. The immunogenicity prediction model training apparatus according to claim 17, wherein the epitope sequence feature encoder and the MHC sequence feature encoder are each based on at least one of: a convolutional neural network, a recurrent neural network, a Transformer model, or a BERT model.

20. A non-transitory computer-readable storage medium, storing computer code which, when executed by at least one processor, causes the at least one processor to at least:
construct an immunogenicity prediction model, the immunogenicity prediction model comprising a binding prediction sub-model, a presentation prediction sub-model, and an immunogenicity prediction sub-model, the binding prediction sub-model being configured to predict binding between an epitope and an MHC, the presentation prediction sub-model being configured to predict cell membrane presentation of a pMHC, and the immunogenicity prediction sub-model being configured to predict immunogenicity of the pMHC; and
input a first sample pair into the immunogenicity prediction model to obtain a sample immunogenicity prediction result output by the immunogenicity prediction model, the first sample pair comprising a first sample epitope sequence and a first sample MHC sequence, and the first sample pair being provided with a first sample immunogenicity label; and
generate a trained immunogenicity prediction model by training the immunogenicity prediction model with the first sample immunogenicity label as a supervisor of the sample immunogenicity prediction result.
